(19) United States
(12) Patent Application Publication
(10) Pub. No.: US 2025/0174717 A1
(43) Pub. Date: May 29, 2025

(54) POLYMER ELECTROLYTES AND BATTERIES WITH THE SAME

(71) Applicant: Toyota Motor Engineering & Manufacturing North America, Inc., Plano, TX (US)

(72) Inventors: Chuhong Wang, Ann Arbor, MI (US); Siwen Wang, Livonia, MI (US); Chen Ling, Troy, MI (US)

(73) Assignees: Toyota Motor Engineering & Manufacturing North America, Inc., Plano, TX (US); Toyota Jidosha Kabushiki Kaisha, Toyota-shi Aichi-ken (JP)

(21) Appl. No.: 18/518,818

(22) Filed: Nov. 24, 2023

(51) Int. Cl. H01M 10/0565; C07D 317/38

(52) U.S. Cl. CPC H01M 10/0565; C07D 317/38; H01M 2300/0082

(57) ABSTRACT

A solid polymer electrolyte includes a charge transfer complex polymer matrix with a polar ring polymer matrix and an ethylene carbonate-based small molecule additive. In some variations, the ethylene carbonate-based small molecule additive is selected from one or more of ethylene carbonate (O═C1OCCO1) and 4-(fluoromethyl)-5-methyl-1,3-dioxolan-2-one (O═C1OC(C)C(CF)O1).

### TECHNICAL FIELD

The present disclosure relates generally to electrolytes for use in secondary batteries, and particularly polymer electrolytes.

### BACKGROUND

Polymer electrolytes combine polymer and ionic salts to provide solid-state (i.e., solid) electrolytes. In addition, and due in part to the flexibility, processability, and possible structural design of polymers, polymer electrolytes provide an attractive alternative to liquid electrolytes. However, traditional polymer electrolytes exhibit reduced ionic conductivity.

The present disclosure addresses issues related to polymer electrolytes, and other issues related to electrolytes.

### SUMMARY

This section provides a general summary of the disclosure, and is not a comprehensive disclosure of its full scope or all of its features.

In one form of the present disclosure, a solid polymer electrolyte includes a charge transfer complex polymer matrix and an ethylene carbonate-based small molecule additive. In some variations, the charge transfer complex polymer matrix comprises a polar ring polymer matrix with electron donors selected from the group consisting of hydroquinone (HQ), tetrathiafulvalene (TTF), phenoxazine (Px),thianthrene (Th), pyrene (Py), and combinations thereof, and electron acceptors selected from the group consisting of benzoquinone (BQ), Cl, 7,7,8,8-tetracyanoquinodimethane (TCNQ), 2,3-Dichloro-5,6-dicyano-1,4-benzoquinone (DDQ), and combinations thereof. In at least one variation, solid polymer electrolyte includes a lithium organic salt, e.g., lithium bis(trifluoromethane)sulfonimide (LiTFSI). And in some variations, the charge transfer complex polymer matrix includes polyphenylene sulfide (PPS) and tetrafluoro-1,4-benzoquinone (TFBQ).

In another form of the present disclosure, a solid polymer electrolyte includes a charge transfer complex polymer matrix with a polar ring polymer matrix and an ethylene carbonate-based small molecule additive. The ethylene carbonate-based small molecule additive is selected from one or both of ethylene carbonate represented by the chemical structure:

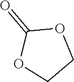

and 4-(fluoromethyl)-5-methyl-1,3-dioxolan-2-one represented by the chemical structure:

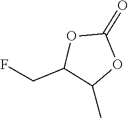

In still another form of the present disclosure, a solid polymer electrolyte includes a charge transfer complex polymer matrix with a polar ring polymer matrix having electron donors selected from one or more of hydroquinone (HQ), tetrathiafulvalene (TTF), phenoxazine (Px),thianthrene (Th), and pyrene (Py), and electron acceptors selected from one or more of benzoquinone (BQ), Cl, 7,7,8,8-tetracyanoquinodimethane (TCNQ), and 2,3-Dichloro-5,6-dicyano-1,4-benzoquinone (DDQ), and an ethylene carbonate-based small molecule additive. The ethylene carbonate-based small molecule additive is selected from one or both of ethylene carbonate represented by the formula:

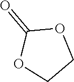

and 4-(fluoromethyl)-5-methyl-1,3-dioxolan-2-one represented by the formula:

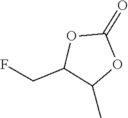

Further areas of applicability and various methods of enhancing the above technology will become apparent from the description provided herein. The description and specific examples in this summary are intended for purposes of illustration only and are not intended to limit the scope of the present disclosure.

### BRIEF DESCRIPTION OF THE DRAWINGS

The present teachings will become more fully understood from the detailed description and the accompanying drawings, wherein:

FIG. 1 illustrates a Li-ion secondary battery with a solid polymer electrolyte;

FIG. 2 illustrates a Li-ion secondary battery with a solid polymer electrolyte according to the teachings of the present disclosure;

FIG. 3 is a graphical plot of simulated Li ion diffusivity as a function of viscosity for a charge transfer complex solid polymer electrolyte with a plurality of different ethylene carbonate-based small molecule additives;

FIG. 4 is a graphical plot of simulated mean square displacement (MSD) as a function of time for a charge transfer complex solid polymer electrolyte with two different ethylene carbonate-based small molecule additives;

FIG. 5A shows a molecular dynamics simulation of a solvation structure for the first Li+ solvation sheath of a charge transfer complex polymer electrolyte without an ethylene carbonate small molecule additive;

FIG. 5B shows a pie chart for the percentage of different Li+ solvates for the charge transfer complex polymer electrolyte without an ethylene carbonate small molecule additive;

FIG. 6A shows a molecular dynamics simulation of a solvation structure for the first Li+ solvation sheath for the charge transfer complex polymer electrolyte with an ethylene carbonate small molecule additive; and

FIG. 6B shows a pie chart for the percentage of different Li+ solvates for the charge transfer complex polymer electrolyte with an ethylene carbonate small molecule additive.

It should be noted that the figures set forth herein are intended to exemplify the general characteristics of metal cation secondary batteries and polymer electrolyte compositions for the purpose of the description of certain aspects. The figures may not precisely reflect the characteristics of any given aspect and are not necessarily intended to define or limit specific forms or variations within the scope of this technology.

### DETAILED DESCRIPTION

The present teachings provide solid polymer electrolytes with enhanced oxidation potential (stability) and metal cation conductivity, i.e., enhanced ionic conductivity. The solid polymer electrolytes according to the teachings of the present disclosure include a charge transfer complex polymer matrix and an ethylene carbonate-based small molecule additive. As used herein, the phrase “small molecule” refers to a molecule with a molecular weight of less than 200 grams per mole. The ethylene carbonate-based small molecule additive, when combined with the charge transfer complex polymer matrix, provides a polymer electrolyte with enhanced ionic conductivity and desired viscosity, i.e., the ethylene carbonate-based small molecule additive does not result in a liquid polymer electrolyte. In addition, the ethylene carbonate-based small molecule additive promotes dissociation of metal cations (e.g., Li⁺) from anions in the solid polymer electrolytes such that conductivity of the metal ions in the solid polymer electrolyte is greater than when the ethylene carbonate-based small molecule additive is not included.

Not being bound by theory, the charge transfer complex (CTC) polymer matrix according to the teachings of the present disclosure includes a polar aromatic ring polymer matrix characterized by intermolecular partial electron transfer between electron donor molecules and electron acceptor molecules. In some variations, the CTC solid polymer matrix includes electron donor molecules selected from hydroquinone (HQ), tetrathiafulvalene (TTF), phenoxazine (Px),thianthrene (Th), and pyrene (Py), and electron acceptors selected from one or more of benzoquinone (BQ), Cl, 7,7,8,8-tetracyanoquinodimethane (TCNQ), 2,3-Dichloro-5,6-dicyano-1,4-benzoquinone (DDQ), and combinations thereof. For example, in some variations the CTC polymer matrix includes or uses polyphenylene sulfide (PPS) to provide electron donor molecules and 1,3,5-tris(4-(4-fluorophenyl)quinolin-2-yl)benzene (TFQB) to provide electron acceptor molecules.

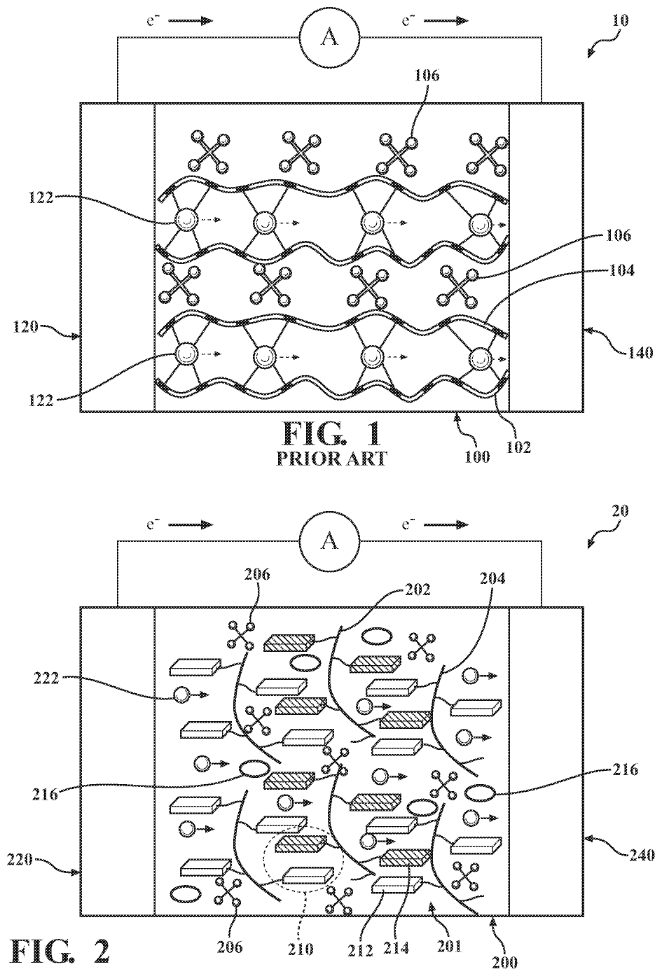

Referring to FIG. 1, a metal ion (e.g., Li ion) secondary battery 10 with a traditional solid polymer electrolyte 100 between an anode 110 (e.g., a Li containing anode) and a cathode 140 (e.g., a carbon containing cathode) is shown. The solid polymer electrolyte 100 includes polymer chains 102, 104, an anion 106, and a metal cation 122. Non-limiting examples of the polymer include polyethylene oxide (PEO), poly(methyl methacrylate) (PMMA), polyvinyl alcohol (PVA), poly(caprolactone) (PCL), poly(vinyl pyrrolidone) (PVP), poly(vinyl chloride) (PVC), poly(vinylidene fluoride) (PVF), and polyimide (PI), among others. In addition, such polymers generally include or have a highly polar motif capable of electron donation.

During discharging of the metal ion secondary battery 10, chemical reactions at the anode 110 create electrons (e⁻) and metal cations 122. The electrons flow through an external circuit 150 to, and are accepted by, the cathode 140 to complete the circuit for the flow of electrons. And the metal ions flow from the anode 120 to the cathode 140 through the solid polymer electrolyte 100 and thereby result in the metal ion secondary battery 10 providing an electric current. Stated differently, chemical energy stored in the metal ion secondary battery 10 is converted to electrical energy. And not being bound by theory, the transport of the metal cations 122 in the solid polymer electrolyte 100 occurs through a mechanism known as segmental motion in which the polymer chains 102, 104 move to allow the passage of metal cations 122.

During charging of the metal ion secondary battery 10, the opposite occurs, i.e., electrical power is applied to the battery such that electrons flow from the cathode 140 to the anode 120 via the external circuit 150 and the metal cations flow from the cathode 140 to the anode 120 through the solid polymer electrolyte 100 such that electrical energy applied to the metal ion secondary battery 10 is converted to stored chemical energy.

Referring to FIG. 2, a metal ion secondary battery 20 with a CTC solid polymer electrolyte 200 according to the teachings of the present disclosure is shown. Similar to the metal ion secondary battery 10, the CTC solid polymer electrolyte is disposed between an anode 220 and a cathode 240 of the metal ion secondary battery 20.

The CTC solid polymer electrolyte 200 includes a charge transfer complex-based polymer matrix 201, an anion 206, and a metal cation 222. The charge transfer complex-based polymer matrix 201 includes a plurality of charge transfer complex groups 210 with electron donor groups 212 and electron acceptor groups 214. In addition, the CTC solid polymer electrolyte 200 includes ethylene carbonate-based small molecules 216 (also referred to herein as “ethylene carbonate-based small molecule additive 216”).

Not being bound by theory, ionic mobility of the metal cations 222 within the charge transfer complex-based polymer matrix 201, without the ethylene carbonate-based small molecule additive 216, is not predominately by segmental motion as with the solid polymer electrolyte 100, but occur vias metal cation hopping along polarized interfaces of the charge transfer complex groups 210 in the charge transfer complex-based polymer matrix 201 as disclosed in the published reference “Charge-Transfer Complexes for Solid-State Li+ Conduction” by Hatakeyama-Soto et al., ACS Appl. Electr4on. Mater. 2020, 2211-2217, and incorporated herein by reference.

In operation, the metal ion secondary battery 20 functions similarly to the metal ion secondary battery 10. However, unlike ionic conductivity in the solid polymer electrolyte 100 and unlike ionic conductivity in the charge transfer complex-based polymer matrix 201 without the ethylene carbonate-based small molecule additive 216, transport of the metal cations 222 in the CTC solid polymer electrolyte 200 is enhanced via dissociation of the metal cations 222 from the anions 206 as discussed in greater detail below.

Referring to FIG. 3, a graphical plot of simulated Li ion (Li⁺) diffusivity versus viscosity for a CTC solid polymer electrolyte with different ethylene carbonate-based small molecules (additives) is shown. Particularly, FIG. 3 illustrates part of a high-throughput screening process to determine or select ethylene carbonate-based small molecules as additives for a CTC solid polymer electrolyte. The CTC solid polymer electrolyte used for the simulations included polyphenylene sulfide (PPS), 1,3,5-tris(4-(4-fluorophenyl)quinolin-2-yl)benzene (TFQB), and lithium bis(trifluoromethanesulfonyl) imide (LiTFSI) (referred to herein as “PPS/TFQB/LiTFSI solid polymer electrolyte”). It should be understood, and as noted above, PPS was included to provide an electron donor group, TFQB was included to provide an electron acceptor group, and LiTFSI was included as a lithium salt that dissociates into Li⁺ cations and TFSI⁻ anions.

The composition of the PPS/TFQB/LiTFSI solid polymer electrolyte with the ethylene carbonate-based small molecule additives (hereafter referred to as “PPS/TFQB/LiTFSI/ECB solid polymer electrolyte(s)”) had a composition, in weight percent, of 56 wt. % PPS/TFQB, 14 wt. % LiTFSI, and 30 wt. % ethylene carbonate-based small molecule additive (i.e., a weight percent ratio of 56/14/30). In addition, a viscosity of 0.007 Pa s for PPS/TFQB/LiTFSI/ECB solid polymer electrolytes was used to prevent or remove the study and simulation of liquid electrolytes. Stated differently, ethylene carbonate-based small molecule additives that resulted in the PPS/TFQB/LiTFSI/ECB solid polymer electrolyte having a viscosity less than 0.007 Pa s were removed from further consideration.

As observed in FIG. 3 approximately 92 differently ethylene carbonate-based small molecule were included in the high-throughput screening process and about 24 of the ethylene carbonate-based small molecule resulted in a simulated PPS/TFQB/LiTFSI/ECB solid polymer electrolyte viscosity greater than 0.007 Pa s. And of these 24 ethylene carbonate-based small molecules, two were chosen for further study. Particularly, ethylene carbonate, represented by the SMTLES formulation as O═C1OCCO1 and the chemical structure shown below, and 4-(fluoromethyl)-5-methyl-1,3-dioxolan-2-one, represented by the SMTLES formulation as O═C1OC(C)C(CF)O1 and the chemical structure shown below, were selected for additional atomistic molecular dynamics simulations.

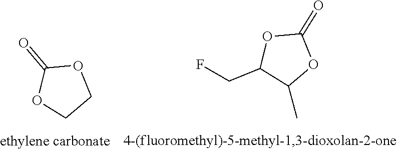

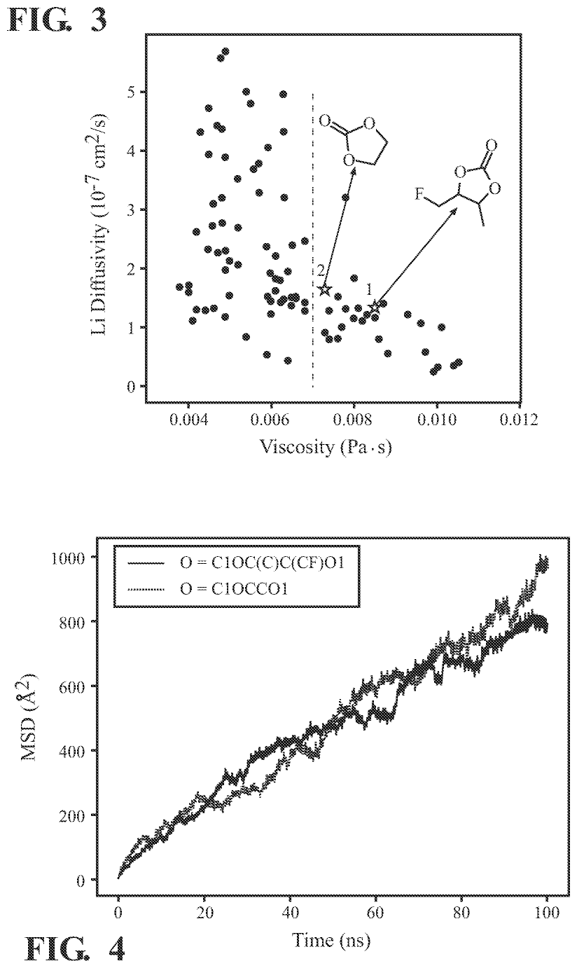

The Li⁺ diffusivities ‘D’ were calculated using the mean squared displacement (MSD) of the Li atoms as illustrated in FIG. 4 and per the relation:

$$D = \frac{〈 {[ {x}_{i} ( t ) - {x}_{i} ( 0 ) ]}^{2} 〉}{6 t} \qquad Eq . 1$$

where ‘x’ is the position of the particle, ‘t is the simulation time, and <⋅> denotes an ensemble average over the particles. In addition, the viscosities ‘η’ of the PPS/TFQB/LiTFSI/ECB solid polymer electrolytes were calculated with the Green-Kubo formulism:

$$η = \frac{V}{{k}_{B} T} {\int }_{0}^{∞} 〈 {P}_{α β} ( t ) \cdot {P}_{α β} ( 0 ) 〉 d t \qquad Eq . 2$$

where V is the system volume, kB is the Boltzmann constant, T is temperature, and P_{αβ}denotes the element αβ of the pressure sensor.

As observed from FIG. 3, the Li⁺ diffusivity ranged from about 0.4×10⁻⁷ cm²/s to about 5.7×10⁻⁷ cm²/s, and the viscosity ranged from about 0.0038 Pa s to about 0.011 Pa s. And as noted above, ethylene carbonate-based small molecules that resulted in a PPS/TFQB/LiTFSI/ECB solid polymer electrolyte with a viscosity less than 0.007 Pa s were removed from further study due to a predicted liquification thereof.

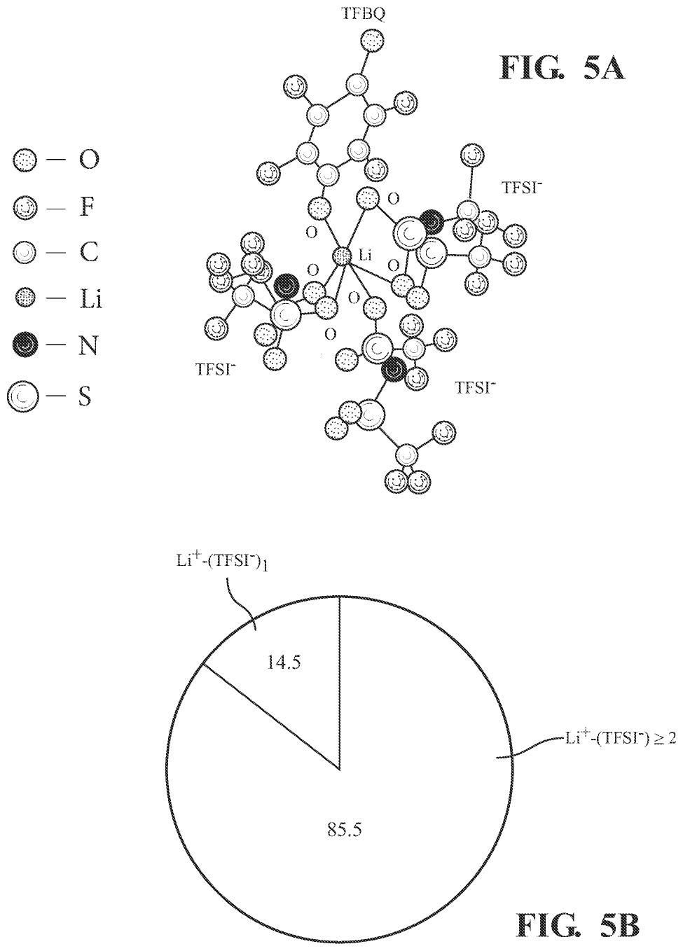

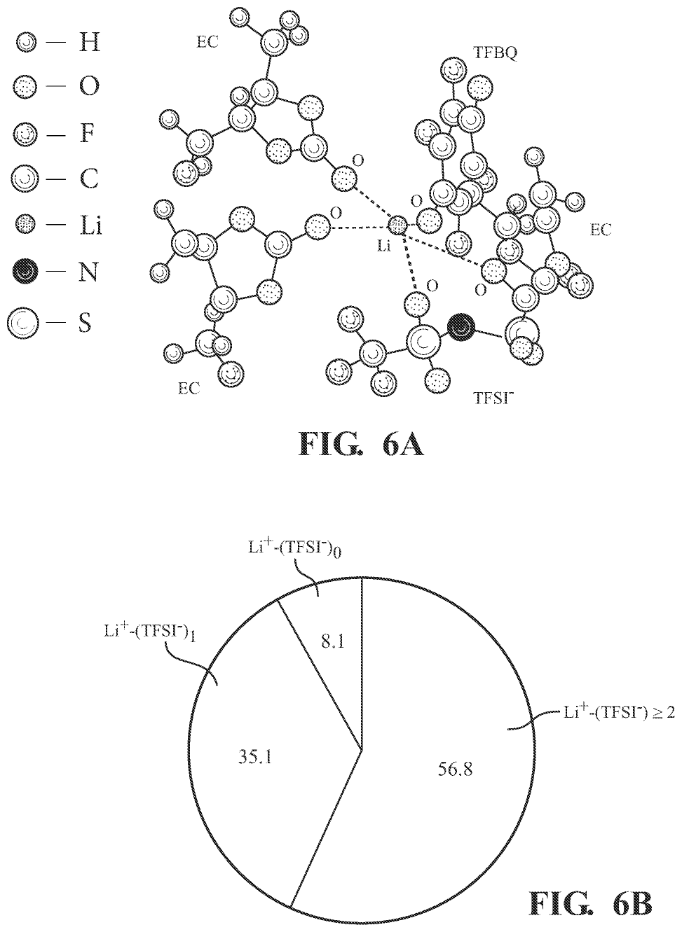

Referring to FIGS. 5A-5B and 6A-6B, the results of additional atomistic molecular dynamics simulations on the PPS/TFBQ/LiTFSI solid polymer electrolyte, with and without the ethylene carbonate small molecule additive, are shown. Particularly, FIG. 5A illustrates the first Li⁺ solvation sheath for a PPS/TFBQ/LiTFSI polymer electrolyte with a weight precent ratio of PPS/TFQB/LiTFSI equal to 80/20, and FIG. 6A illustrates the first Li⁺ solvation sheath for the PPS/TFBQ/LiTFSI/ECB solid polymer electrolyte with the ethylene carbonate small molecule additive and the above noted weight percent ratio equal to 56/14/30. And as observed from FIGS. 5A, in the PPS/TFBQ/LiTFSI solid polymer electrolyte the Li⁺ is bound to one electron acceptor group (i.e., a TFBQ molecule) and three (3) TFSI⁻ cations. However, FIG. 6A illustrates Li⁺ in the PPS/TFBQ/LiTFSI/ECB solid polymer electrolyte is bound to one electron acceptor group (TFBQ), one TFSI⁻ anion, and three (3) ethylene carbonate small molecules. Accordingly, FIGS. 5A and 6A illustrate that the ethylene carbonate small molecule additive reduces the number of bounds between Li+ cations and TFSI⁻ anions. Stated differently, the ethylene carbonate small molecule additive reduces the association of Li⁺ with TFSI⁻ anion clusters.

And with reference to FIGS. 5B and 6B, FIG. 5B illustrates that the PPS/TFBQ/LiTFSI solid polymer electrolyte exhibits 14.5% Li⁺ cations bound with a single TFSI⁻ anion (labeled Li⁺-(TFSI⁻)₁) and 85.5% Li+ cations bound with two or more TFSI⁻ anions (labeled Li⁺-(TFSI⁻)_{≥2}). In contrast, FIG. 6B illustrates that the PPS/TFBQ/LiTFSI/ECB solid polymer electrolyte exhibits 8.1% Li⁺ cations not bound with a TFSI⁻ anion (labeled Li⁺-(TFSI⁻)₀), 35.1% Li⁺ cations bound with a single TFSI⁻ anion (labeled Li⁺-(TFSI⁻)₁), and only 56.8% Li⁺ cations bound with two or more TFSI-anions (labeled Li⁺-(TFSI⁻)_{≥2}). Accordingly, FIGS. 5A-6B illustrate that the inclusion of the ethylene carbonate in the PPS/TFBQ/LiTFSI polymer electrolyte reduces the aggregation of anions around the solvation sheath of Li⁺ ions and thereby creates a more favorable environment for metal cation movement and enhanced ionic conductivity.

The foregoing description relates to what are presently considered to be the most practical embodiments. It is to be understood, however, that the disclosure is not to be limited to these embodiments but, on the contrary, is intended to cover various modifications and equivalent arrangements included within the spirit and scope of the appended claims, which scope is to be accorded the broadest interpretation so as to encompass all such modifications and equivalent structures as is permitted under the law.

For example, and even though FIGS. 5A-6B illustrate the effect of the ethylene carbonate small molecule on the ionic conductivity of the PPS/TFBQ/LiTFSI solid polymer electrolyte, it should be understood that the 4-(fluoromethyl)-5-methyl-1,3-dioxolan-2-one would have the same effect. In addition, and with the teachings of the present disclosure embodying the enhanced ionic conductivity of a metal cation in a CTC solid polymer matrix with ethylene carbonate-based small molecule additives, it should be understood that CTC solid polymer matrices and ethylene-based small molecules not specifically discussed herein are included in the teachings and scope of the present disclosure.

The preceding description is merely illustrative in nature and is in no way intended to limit the disclosure, its application, or uses. As used herein, the phrase at least one of A, B, and C should be construed to mean a logical (A or B or C), using a non-exclusive logical “or.” It should be understood that the various steps within a method may be executed in different order without altering the principles of the present disclosure. Disclosure of ranges includes disclosure of all ranges and subdivided ranges within the entire range.

The headings (such as “Background” and “Summary”) and sub-headings used herein are intended only for general organization of topics within the present disclosure, and are not intended to limit the disclosure of the technology or any aspect thereof. The recitation of multiple forms or variations having stated features is not intended to exclude other forms or variations having additional features, or other forms or variations incorporating different combinations of the stated features.

As used herein the term “about” when related to numerical values herein refers to known commercial and/or experimental measurement variations or tolerances for the referenced quantity. In some variations, such known commercial and/or experimental measurement tolerances are +/−10% of the measured value, while in other variations such known commercial and/or experimental measurement tolerances are +/−5% of the measured value, while in still other variations such known commercial and/or experimental measurement tolerances are +/−2.5% of the measured value. And in at least one variation, such known commercial and/or experimental measurement tolerances are +/−1% of the measured value.

As used herein, the terms “comprise” and “include” and their variants are intended to be non-limiting, such that recitation of items in succession or a list is not to the exclusion of other like items that may also be useful in the devices and methods of this technology. Similarly, the terms “can” and “may” and their variants are intended to be non-limiting, such that recitation that a form or variation can or may comprise certain elements or features does not exclude other forms or variations of the present technology that do not contain those elements or features.

The broad teachings of the present disclosure can be implemented in a variety of forms. Therefore, while this disclosure includes particular examples, the true scope of the disclosure should not be so limited since other modifications will become apparent to the skilled practitioner upon a study of the specification and the following claims. Reference herein to one aspect, or various aspects means that a particular feature, structure, or characteristic described in connection with a form or variation is included in at least one form or variation. The appearances of the phrase “in one variation” or “in one form” (or variations thereof) are not necessarily referring to the same form or variation. It should be also understood that the various method steps discussed herein do not have to be carried out in the same order as depicted, and not each method step is required in each form or variation.

The foregoing description of the forms or variations has been provided for purposes of illustration and description. It is not intended to be exhaustive or to limit the disclosure. Individual elements or features of a particular form or variation are generally not limited to that particular form or variation, but, where applicable, are interchangeable and can be used in a selected form or variation, even if not specifically shown or described. The same may also be varied in many ways. Such variations should not be regarded as a departure from the disclosure, and all such modifications are intended to be included within the scope of the disclosure.

While particular forms or variations have been described, alternatives, modifications, variations, improvements, and substantial equivalents that are or may be presently unforeseen may arise to applicants or others skilled in the art. Accordingly, the appended claims as filed and as they may be amended, are intended to embrace all such alternatives, modifications variations, improvements, and substantial equivalents.

What is claimed is:

1. A solid polymer electrolyte comprising:
a charge transfer complex polymer matrix; and
an ethylene carbonate-based small molecule additive.

2. The solid polymer electrolyte according to claim 1, wherein the charge transfer complex polymer matrix comprises a polar ring polymer matrix.

3. The solid polymer electrolyte according to claim 2, wherein the charge transfer complex polymer matrix comprises electron donors selected from the group consisting of hydroquinone (HQ), tetrathiafulvalene (TTF), phenoxazine (Px),thianthrene (Th), pyrene (Py), and combinations thereof.

4. The solid polymer electrolyte according to claim 3, wherein the charge transfer complex polymer matrix comprises electron acceptors selected from the group consisting of benzoquinone (BQ), Cl, 7,7,8,8-tetracyanoquinodimethane (TCNQ), 2,3-Dichloro-5,6-dicyano-1,4-benzoquinone (DDQ), and combinations thereof.

5. The solid polymer electrolyte according to claim 4 further comprising a lithium organic salt.

6. The solid polymer electrolyte according to claim 5, wherein the lithium organic salt is lithium bis(trifluoromethane)sulfonimide (LiTFSI).

7. The solid polymer electrolyte according to claim 6, wherein the charge transfer complex polymer matrix comprises polyphenylene sulfide (PPS) and tetrafluoro-1,4-benzoquinone (TFBQ).

8. The solid polymer electrolyte according to claim 7, wherein the ethylene carbonate-based small molecule additive has a molecular weight less than or equal to 150 grams per mole.

9. The solid polymer electrolyte according to claim 8, wherein the ethylene carbonate-based small molecule additive is selected from the group consisting of ethylene carbonate represented by the chemical structure: 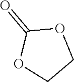
4-(fluoromethyl)-5-methyl-1,3-dioxolan-2-one represented by the chemical structure: 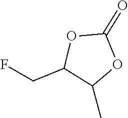
and combinations thereof.

10. A solid polymer electrolyte comprising:
a charge transfer complex polymer matrix comprising a polar ring polymer matrix; and
an ethylene carbonate-based small molecule additive selected from the group consisting of ethylene carbonate represented by the chemical structure: 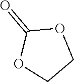
4-(fluoromethyl)-5-methyl-1,3-dioxolan-2-one represented by the chemical structure: 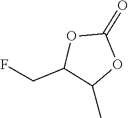
and combinations thereof.

11. The solid polymer electrolyte according to claim 1, wherein the charge transfer complex polymer matrix comprises a polar ring polymer matrix with electron donors selected from the group consisting of hydroquinone (HQ), tetrathiafulvalene (TTF), phenoxazine (Px),thianthrene (Th), pyrene (Py), and combinations thereof.

12. The solid polymer electrolyte according to claim 11, wherein the charge transfer complex polymer matrix comprises electron acceptors selected from the group consisting of benzoquinone (BQ), Cl, 7,7,8,8-tetracyanoquinodimethane (TCNQ), 2,3-Dichloro-5,6-dicyano-1,4-benzoquinone (DDQ), and combinations thereof.

13. The solid polymer electrolyte according to claim 11 further comprising a lithium organic salt.

14. The solid polymer electrolyte according to claim 13, wherein the lithium organic salt is lithium bis(trifluoromethane)sulfonimide (LiTFSI).

15. The solid polymer electrolyte according to claim 14, wherein the charge transfer complex polymer matrix comprises polyphenylene sulfide (PPS) and tetrafluoro-1,4-benzoquinone (TFBQ).

16. A solid polymer electrolyte comprising:
a charge transfer complex polymer matrix comprising a polar ring polymer matrix with electron donors selected from the group consisting of hydroquinone (HQ), tetrathiafulvalene (TTF), phenoxazine (Px),thianthrene (Th), pyrene (Py), and combinations thereof, and electron acceptors selected from the group consisting of benzoquinone (BQ), Cl, 7,7,8,8-tetracyanoquinodimethane (TCNQ), 2,3-Dichloro-5,6-dicyano-1,4-benzoquinone (DDQ), and combinations thereof, and
an ethylene carbonate-based small molecule additive selected from the group consisting of ethylene carbonate represented by the chemical structure: 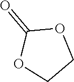
4-(fluoromethyl)-5-methyl-1,3-dioxolan-2-one represented by the chemical structure: 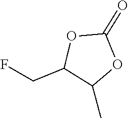
and combinations thereof.

17. The solid polymer electrolyte according to claim 16 further comprising a lithium organic salt.

18. The solid polymer electrolyte according to claim 17, wherein the lithium organic salt is lithium bis(trifluoromethane)sulfonimide (LiTFSI).

19. The solid polymer electrolyte according to claim 18, wherein the charge transfer complex polymer matrix comprises polyphenylene sulfide (PPS) and tetrafluoro-1,4-benzoquinone (TFBQ).

20. The solid polymer electrolyte according to claim 19, wherein the ethylene carbonate-based small molecule additive has a molecular weight less than or equal to 150 grams per mole.
